(19) United States
(12) Patent Application Publication
(10) Pub. No.: US 2018/0049722 A1
(43) Pub. Date: Feb. 22, 2018

(54) ULTRASONIC DIAGNOSIS SYSTEM

(71) Applicant: Hitachi, Ltd., Tokyo (JP)

(72) Inventor: Takaya UNO, Tokyo (JP)

(73) Assignee: Hitachi, Ltd., Tokyo (JP)

(21) Appl. No.: 15/561,000

(22) PCT Filed: Mar. 23, 2016
(86) PCT No.: PCT/JP2016/059219

§ 371 Date: Sep. 22, 2017

(30) Foreign Application Priority Data
Mar. 25, 2015 (JP) 2015-062466

(51) Int. Cl. A61B 8/00; H04L 29/08; H04L 9/08; H04W 12/06

(52) U.S. Cl. CPC A61B 8/56; H04L 67/1074; H04W 12/06; A61B 8/4427; H04L 9/0819; A61B 8/4433

(57) ABSTRACT

Provided is an ultrasonic diagnosis system in which a front-end (FE) device and a back-end (BE) device are set to a docking state, i.e., a wired connection state. While in this state, wired communication is used prior to wireless communication to exchange (pairing) key information (encryption keys, link keys) for performing exclusive wireless communication between the devices. The key information is stored in non-volatile memory in each device. After pairing, exclusive wireless communication is carried out between the devices using the key information each time that a separation state is formed.

### TECHNICAL FIELD

The present disclosure relates to an ultrasound diagnostic apparatus, and in particular, to an ultrasound diagnostic apparatus formed from a plurality of transportable devices.

### BACKGROUND

An ultrasound diagnostic system is an apparatus which forms an ultrasound image based on a reception signal obtained by transmission and reception of ultrasound to and from a living body. When an ultrasound diagnostic system is formed from a plurality of devices (units, modules) independent from each other, typically, the plurality of devices are used in a separated state or in a docked state. In the separated state, the plurality of devices are electrically connected to each other through a wireless communication scheme. In the docked state, the plurality of devices are connected by a wired communication scheme. The latter state includes a state where two devices are connected by a cable.

Patent Literature 1 discloses an ultrasound diagnostic system having a first housing and a second housing. Patent Literature 2 discloses an ultrasound diagnostic system having a front-end device and a back-end device. In an ultrasound diagnostic system disclosed in Patent Literature 3, a device body and an ultrasound probe are connected wirelessly.

### CITATION LIST

### Patent Literature

Patent Literature 1: JP 2011-5241 A

Patent Literature 2: JP 2008-114065 A

Patent Literature 3: JP 2011-87841 A

### SUMMARY

### Technical Problem

When a plurality of devices forming the ultrasound diagnostic system can selectively take a docked state or a separated state, the data is exchanged between the devices through a wireless communication in the separated state. In this case, from the viewpoint of security, a situation where the individual device is unpreparedly connected to other unspecified devices must be reliably avoided. In addition, in order to establish an exclusive wireless communication, if a complex user operation is requested such as a code input every time the devices are set in the separated state, the operability is reduced. In the ultrasound diagnostic system, the plurality of devices which are in a connection relationship are fixed from the beginning, and thus, it is desired to reliably and simply establish the wireless communication among the plurality of devices, based on this fact.

An advantage of the present disclosure lies in enabling secure wireless communication among a plurality of devices in an ultrasound diagnostic system. An alternative advantage of the present disclosure lies in reliable and quick establishment of exclusive wireless communication among a plurality of devices every time the plurality of devices are set in a separated state. Another alternative advantage of the present disclosure lies in utilizing wired communication for wireless communication.

### Solution to Problem

According to one aspect of the present disclosure, there is provided an ultrasound diagnostic system comprising: a first device and a second device that operate for ultrasound diagnosis, wherein the first device and the second device selectively take one of a wired connection state and a wireless connection state, the first device comprises a first wired communicator and a first wireless communicator, the second device comprises a second wired communicator that executes wired communication with the first wired communicator, and a second wireless communicator that executes wireless communication with the first wireless communicator, and, in the wired connection state, pairing is executed utilizing the wired communication, in which key information necessary for exclusive wireless communication between the first device and the second device is exchanged.

In the ultrasound diagnostic system, the first device and the second device can selectively take a wired connection state (typically, the docked state), and a wireless communication state (typically, the separated state). In the wired connection state, information necessary for future wireless communication is exchanged utilizing wired communication between the first device and the second device. That is, pairing is executed. The exchanged information is key information for executing exclusive wireless communication between the first device and the second device. The key information acquired by the first device and the key information acquired by the second device may be the same or different. The key information is, for example, an encryption key and a link key (PIN (Personal Identification Number) code). As a part of the key information, or separately from the key information, data for identifying or authenticating the other party (for example, a device ID, a network ID, and an address) may be exchanged. Because the key information is exchanged utilizing the wired communication, and not through the wireless communication, that is, because preparation for the wireless communication can be executed while actually checking and identifying the two devices assuming a physical connection state (typically, docking between the devices), it becomes possible to accurately and easily execute the preparation. The load on the user due to the pairing work can be reduced or even removed. The pairing work is desirably executed at the time of shipping from the factory (at the time of initial setup), and executed again at the time of maintenance (at the time of setup another time). The pairing may also be understood as prior authentication for wireless communication.

According to another aspect of the present disclosure, in the pairing, first key information for authenticating the first device at the second device is sent from the first device to the second device, the first key information is stored in a non-volatile storage in the first device, second key information for authenticating the second device at the first device is sent from the second device to the first device, and the second key information is stored in a non-volatile memory in the second device.

According to another aspect of the present disclosure, a plurality of wireless communication sessions are established in parallel to each other between the first wireless communicator and the second wireless communicator, the first key information includes a plurality of data for establishing the plurality of wireless communication sessions, and the second key information includes a plurality of data for establishing the plurality of wireless communication sessions.

According to another aspect of the present disclosure, the plurality of wireless communication sessions include wireless communication for transferring ultrasound reception data from the first device to the second device, and wireless communication for transferring control data from the second device to the first device. According to another aspect of the present disclosure, the first device is a front-end device having an ultrasound transmission and reception circuit, and the second device is a back-end device having an ultrasound image display. The first device may be a device body and the second device may be a probe. Alternatively, the above-described technique may be applied to individual devices in a system having a first device, a second device, and a third device, in a state where the three devices are docked.

According to another aspect of the present disclosure, the first device and the second device are set in a freeze state during a process of changing from the separated state to the docked state, before the docked state is established. According to such a configuration, it is possible to prevent instability in the operation of the ultrasound diagnostic system. According to another aspect of the present disclosure, the first device and the second device are set in a freeze state during a process of changing from a docked state to a separated state. According to such a configuration, it is possible to prevent instability in the operation of the ultrasound diagnostic system. In addition, the security can be improved. The freeze state is released by a freeze release operation by the user.

According to another aspect of the present disclosure, there is provided a method comprising: setting a first device and a second device for ultrasound diagnosis in a docked state; in the docked state, executing pairing, utilizing wired communication, to exchange key information for executing exclusive wireless communication between the first device and the second device; and, after the pairing, establishing exclusive wireless communication using the key information between the first device and the second device every time the first device and the second device are set in a separated state. The method may be realized by a control program for the first device and a control program for the second device. The programs are supplied to the devices via a recording medium, or via a network. Alternatively, the programs are pre-installed in a storage device in each device.

### Advantageous Effects of Invention

According to various aspects of the present disclosure, secure wireless communication can be executed among a plurality of devices forming an ultrasound diagnostic system, the wireless communication can be reliably and quickly established every time the plurality of devices are set in the separated state, or the wired communication can be utilized for the wireless communication.

### BRIEF DESCRIPTION OF DRAWINGS

FIG. 1 is a conceptual diagram showing an ultrasound diagnostic system according to an embodiment of the present disclosure.

FIG. 2 is a perspective diagram of an ultrasound diagnostic system in a separated state.

FIG. 3 is a perspective diagram of an ultrasound diagnostic system in a docked state.

FIG. 4 is a block diagram of a front-end device.

FIG. 5 is a block diagram of a back-end device.

FIG. 6 is a diagram showing a communication scheme in a docked state and a communication scheme in a separated state.

FIG. 7 is a diagram showing exchange of authentication data utilizing wired communication, for wireless communication.

FIG. 8 is a flowchart showing an example operation during pairing.

FIG. 9 is a flowchart showing an example operation when wireless communication is established.

### DESCRIPTION OF EMBODIMENTS

An embodiment of the present disclosure will now be described with reference to the drawings.

### (1) Ultrasound Diagnostic System

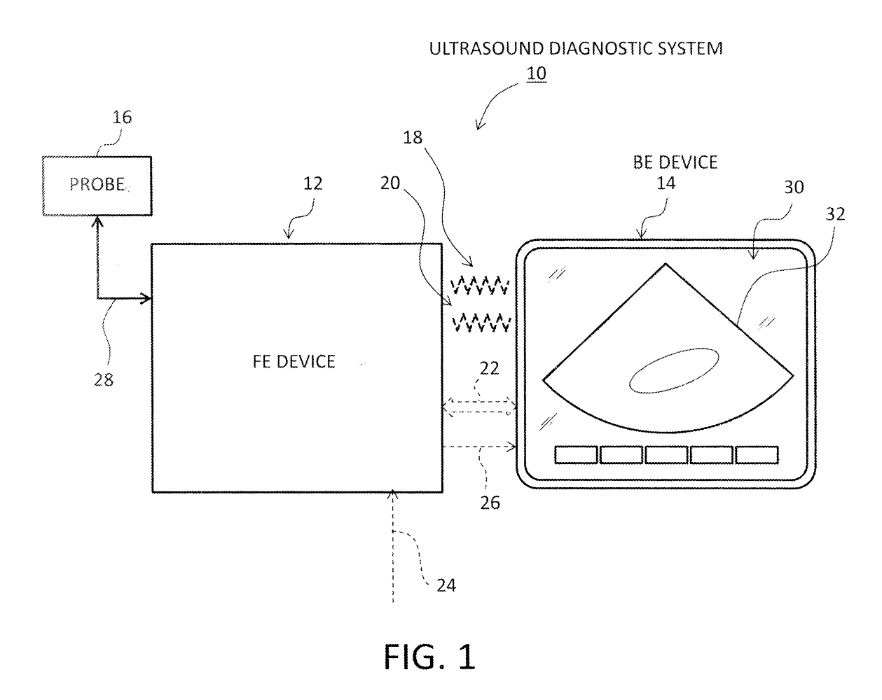

FIG. 1 schematically shows a structure of an ultrasound diagnostic system according to an embodiment of the present disclosure. An ultrasound diagnostic system 10 is a medical device used in a medical institution such as hospitals, and executes ultrasound diagnosis on a subject (living body). The ultrasound diagnostic system 10 is roughly formed from a front-end (FE) device 12, a back-end (BE) device 14, and a probe 16. The FE device 12 is a device closer to the living body, and the BE device 14 is a device which is farther away from the living body. The FE device 12 and the BE device 14 are provided as separate structures, and each forms a transportable device. The FE device 12 and the BE device 14 can be operated in a separated state in which the devices are detached from each other, and can also be operated in a docked state where the devices are put together. FIG. 1 shows the separated state.

The probe 16 is a transmitting and receiving device which transmits and receives ultrasound in a state where the probe 16 contacts a surface of the living body. The probe 16 comprises a 1-D array transducer having a plurality of transducer elements arranged in a liner shape or an arc shape. An ultrasound beam is formed by the array transducer, and is repeatedly electrically scanned. For each electric scan, a beam scan plane is formed in the living body. As the electric scanning scheme, an electric linear scan scheme, an electric sector scan scheme, and the like are known. Alternatively, a 2-D array transducer which can form a three-dimensional echo data capturing space may be provided in place of the 1-D array transducer. In the example configuration of FIG. 1, the probe 16 is connected to the FE device 12 via a cable 28. The probe 16 may alternatively be connected to the FE device 12 by wireless communication. In this case, a wireless probe is used. Alternatively, in a state where a plurality of probes are connected to the FE device 12, a probe to be actually used may be selected from among the connected probes. Alternatively, a probe which is to be inserted into a body cavity may be connected to the FE device 12.

In the separated state shown in FIG. 1, the FE device 12 and the BE device 14 are electrically connected to each other by a wireless communication scheme. In the present embodiment, the devices are connected to each other by a first wireless communication scheme and a second wireless communication scheme. In FIG. 1, a wireless communication path 18 using the first wireless communication scheme and a wireless communication path 20 using the second wireless communication scheme are explicitly shown. The first wireless communication scheme is faster compared to the second wireless communication scheme, and in the present embodiment, ultrasound reception data is transferred from the FE device 12 to the BE device 14 using the first wireless communication scheme. That is, the first wireless communication scheme is used for data transfer. The second wireless communication scheme is a communication scheme which is slower and simpler than the first wireless transfer scheme, and in the present embodiment, control signals are transferred from the BE device 14 to the FE device 12 using the second wireless communication scheme. In other words, the second wireless communication scheme is used for control.

In the docked state in which the FE device 12 and the BE device 12 are physically combined, the FE device 12 and the BE device 14 are electrically connected to each other by a wired communication scheme. Compared to the above-described two wireless communication schemes, the wired communication scheme is significantly faster. In FIG. 1, a wired communication path 22 formed between the two devices 12 and 14 is shown. A power supply path 26 is for supplying direct current electric power from the FE device 12 to the BE device 14 in the docked state. The electric power is used for the operations of the BE device 14, and is also used for charging a battery in the BE device 14.

Reference numeral 24 represents a DC power supply line supplied from an AC adaptor (AC/DC converter). The AC adapter is connected to the FE device 12 as necessary. The FE device 12 also has a battery built therein, and can operate using the battery as a power supply. The FE device 12 has a box-like form, as will be described later. A structure and an operation of the FE device 12 will be described later in detail.

Meanwhile, in the present embodiment, the BE device 14 has a tablet form or a flat plate form. The BE device 14 thus has a basic structure similar to a typical tablet computer. The BE device 14 is equipped with various dedicated software for ultrasound diagnosis, including an operation control program, an image processing program, or the like. The BE device 14 has a display panel 30 with a touch sensor. The display panel 30 functions as a user interface having functions of both an inputter and a display. In FIG. 1, a B-mode tomographic image is displayed on the display panel 30 as an ultrasound image. The user executes various inputs using a group of icons displayed on the display panel 30. It is also possible to execute a slide operation, an enlarging operation, or the like on the display panel 30.

Depending on the diagnosis usage and preferences of the inspector, or the like, the ultrasound diagnosis system 10 can be operated in a usage form selected from the separated state and the docked state. Thus, an ultrasound diagnostic system can be provided having a superior usability.

In order to prevent the operations of the ultrasound diagnostic system 10 becoming unstable or inappropriate during a state change, in the present embodiment, during the state change, control is executed to forcefully put the ultrasound diagnostic system 10 into a freeze state. Specifically, in the process of transferring from the separated state to the docked state, the FE device 12 and the BE device 14 each judge a timing immediately before the docking, based on a radio wave intensity or reception state indicating a distance between the FE device 12 and the BE device 14, and control is executed in each of the devices 12 and 14 to transfer the operation state to the freeze state according to the judgment. After the docked state is established and an operation to release freeze by the inspector, the freeze states of the devices 12 and 14 are released. In the process of transfer from the docked state to the separated state, the FE device 12 and the BE device 14 individually detect that the devices are set in the separated state by line detection or other methods, and the devices are set in the freeze state. Then, after an operation to release the freeze, the freeze states of the devices 12 and 14 are released.

The BE device 14 may be separately connected to an in-hospital LAN via a wireless communication scheme or a wired communication scheme. The communication paths for these communications are not shown in FIG. 1. Alternatively, the BE device 14 (or the FE device 12) may be separately connected to other dedicated devices (for example, a remote controller) which function for the ultrasound diagnosis, by a wireless communication scheme or a wired communication scheme.

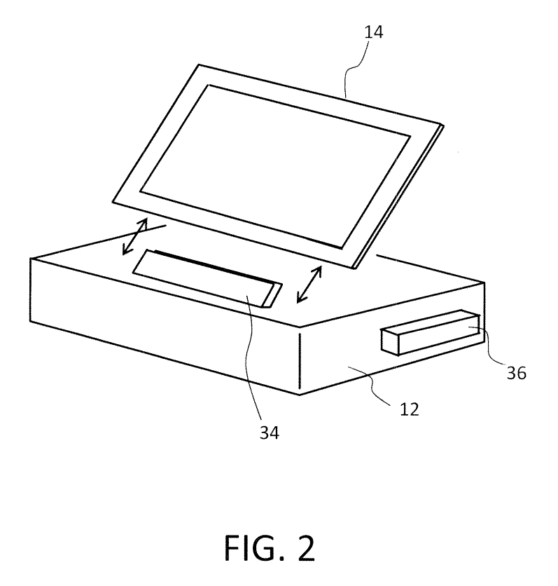

FIG. 2 shows a separated state. The FE device 12 is placed, for example, on a desk. The FE device 12 has a holder 34 having an insertion port (slot). The holder 34 has a hinge mechanism, and can rotate about a horizontal axis. On a certain side surface of the FE device 12, a connector provided on an end of a probe cable is attached. Alternatively, a compartment for housing the probe or the like may be formed inside the FE device 12. Such a configuration is convenient when the ultrasound diagnostic system is transported, and can protect the probe. In FIG. 2, the BE device 14 is separated from the FE device 12, and the BE device 14 can be moved farther away from the FE device 12, so long as the wireless communication is possible.

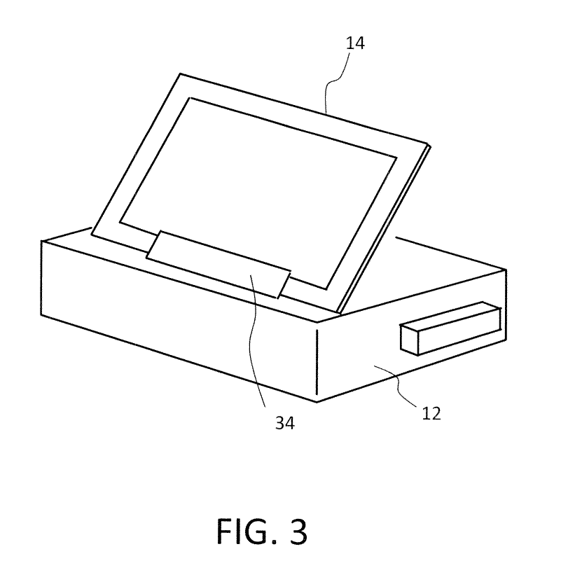

FIG. 3 shows a docked state. A lower end of the BE device is inserted into the insertion port of the holder 34. In this inserted state, the FE device 12 and the BE device 14 are in a wired connection state. In other words, the devices are connected by a wired LAN, and are connected by a wired power supply line. In the docked state, an angle of the BE device 14 can be arbitrarily varied, to change the orientation thereof. The BE device 14 may be tilted completely to the back surface side thereof (upper surface side of the FE device 12).

### (2) Front-End Device

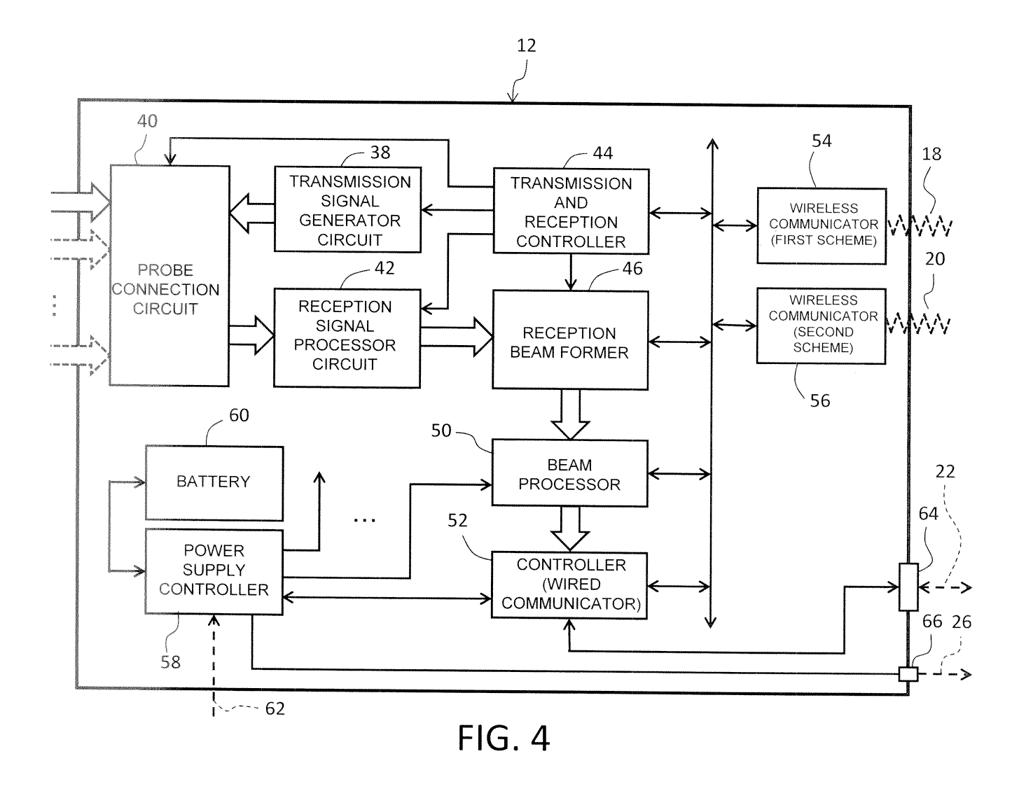

FIG. 4 is a block diagram of the FE device 12. Each block in FIG. 4 is formed with hardware such as a processor, an electronic circuit, or the like. A transmission signal generator circuit 38 is a circuit which supplies a plurality of transmission signals in parallel to each other to the plurality of transducer elements in the probe via a probe connection circuit 40. With the supply of the signals, a transmission beam is formed at the probe. When a reflection wave from within the living body is received by the plurality of transducer elements, a plurality of reception signals are output from the transducer elements, and are input to a reception signal processor circuit 42 via the probe connection circuit 40. The reception signal processor circuit 42 includes a plurality of pre-amplifiers, a plurality of amplifiers, and a plurality of A/D converters, and the like. A plurality of digital reception signals which are output from the reception signal processor circuit 42 are sent to a reception beam former 46. The reception beam former 46 applies phasing addition on the plurality of digital reception signals, and outputs beam data as a signal after the phasing addition. The beam data corresponds to the reception beam, and comprises a plurality of echo data arranged in a depth direction. Reception frame data is formed by a plurality of beam data obtained by one electric scan.

A transmission and reception controller 44 controls the transmission signal generation and the reception signal process based on transmission and reception control data sent from the BE device. A beam processor 50 is a circuit which applies various data processes such as wave detection, logarithmic conversion, correlation, or the like to individual beam data which are input in a time sequential order. A control unit 52 controls an overall operation of the FE device 12. In addition, the control unit 52 executes control for wired transfer or wireless transfer, to the BE device, of beam data which are sequentially sent from the beam processor. In the present embodiment, the control unit 52 also functions as a wired communicator. A wireless communicator 54 is a module for executing communication in the first wireless communication scheme. A wireless communicator 56 is a module for executing communication in the second wireless communication scheme. Reference numeral 18 shows the wireless communication path according to the first wireless communication scheme, and reference numeral 20 shows the wireless communication path according to the second wireless communication scheme. The paths are bidirectional transfer paths, but in the present embodiment, a large amount of reception data is transferred from the FE device 12 to the BE device using the former, and control signals are transferred from the BE device to the FE device 12 using the latter. Reference numeral 64 shows a wired communication terminal, and the wired communication path 22 is connected thereto. Reference numeral 66 shows a power supply terminal, and the power supply line 26 is connected thereto. As described above, the power supply line 26 is a line for supplying the direct current electric power from the FE device 12 to the BE device.

A battery 60 is, for example, a lithium ion type battery, and charging and discharging therein are controlled by a power supply controller 58. At the time of battery drive, the electric power from the battery 60 is supplied via the power supply controller 58 to the circuits in the FE device 12. Reference numeral 62 shows a power supply line at the time when the AC adaptor is connected. At the time when the AC adaptor is connected, external electric power is supplied to the circuits in the FE device 12 by an action of the power supply controller 58. In this process, if the charge of the battery 60 is less than 100%, the battery 60 is charged using the external electric power.

During an ultrasound diagnosis operation (during transmission and reception), the FE device 12 repeatedly executes supply of a plurality of transmission signals to the probe and processing of a plurality of reception signals obtained thereafter, in accordance with control from the side of the BE device. The time sequential beam data obtained as a result of this process is consecutively transferred to the BE device by the wireless communication in the separated state and by the wired communication in the docked state. In this process, individual beam data is converted into a plurality of packets, and individual beam data is transferred by a so-called packet transfer scheme.

As the operation mode, in addition to the B mode, there are known various modes such as a CFM mode, an M mode, and a D mode (a PW mode and a CW mode). Alternatively, transmission and reception processes for harmonic imaging and elasticity information imaging may be executed. In FIG. 1, circuits such as a living body signal input circuit are not shown.

### (3) Back-End Device

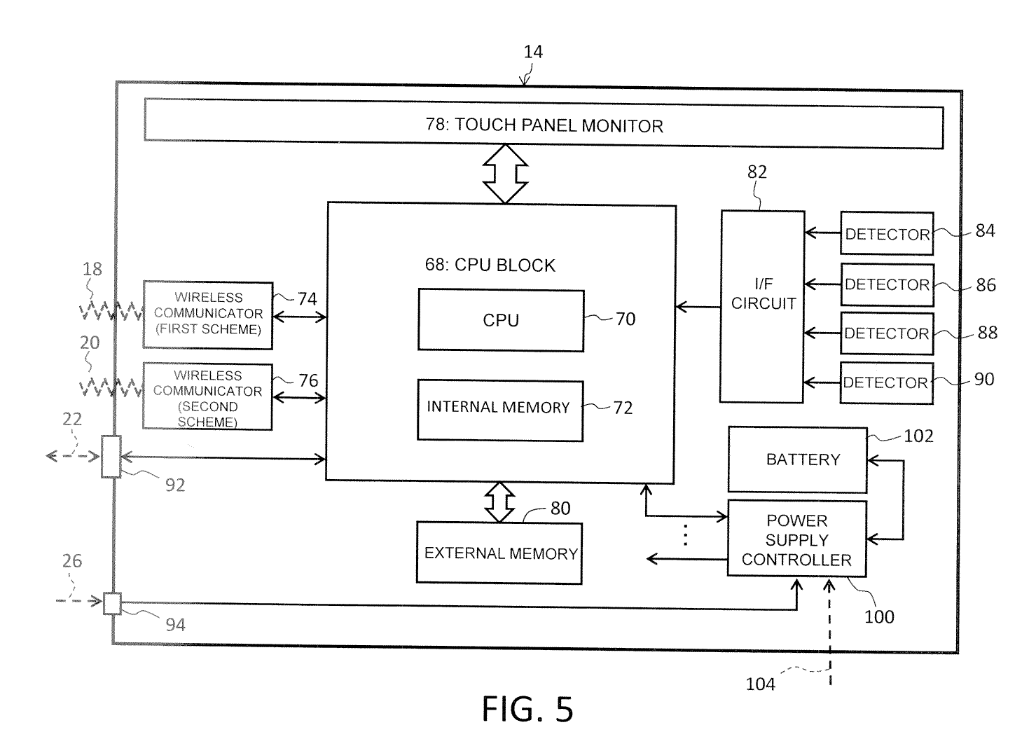

FIG. 5 is a block diagram of the BE device 14. In FIG. 5, each block shows hardware such as a processor circuit, a memory, or the like. A CPU block 68 comprises a CPU 70, an internal memory 72, or the like. The internal memory 72 functions as a working memory or a cache memory. An external memory 80 connected to the CPU block 68 stores an OS, various control programs, various processing programs, or the like. The latter includes a scan conversion process program. The external memory 80 also functions as a cine-memory having a ring buffer structure. The cine-memory may alternatively be formed in the internal memory 72.

The CPU block 68 generates display frame data by a scan conversion process based on a plurality of beam data. The display frame data forms an ultrasound image (for example, a tomographic image). The process is consecutively executed, to generate a video image. The CPU block 68 applies various processes to the beam data or the image for ultrasound image display. In addition, the CPU block 68 controls the operation of the BE device 14, and controls the ultrasound diagnostic system as a whole.

A touch panel monitor (display panel) 78 functions as an input device and a display device. Specifically, the touch panel monitor 78 includes a liquid crystal display and a touch sensor, and functions as a user interface. On the touch panel monitor 78, a display image including the ultrasound image is displayed, and various buttons (icons) for operation are displayed.

A wireless communicator 74 is a module for the wireless communication according to the first wireless communication scheme. A wireless communication path for this wireless communication is shown with reference numeral 18. A wireless communicator 76 is a module for the wireless communication according to the second wireless communication scheme. A wireless communication path for this wireless communication is shown with reference numeral 20. The CPU block 68 also has a function to execute wired communication according to a wired communication scheme. In the docked state, a wired communication line is connected to a wired communication terminal 92. In addition, the power supply line 26 is connected to a power supply terminal 94.

A plurality of detectors 84˜90 are connected to the CPU block 68 via an I/F circuit 82. The detectors include an illumination sensor, a proximity sensor, a temperature sensor, or the like. Alternatively, a module such as a GPS may be connected to the I/F circuit 82. The I/F circuit 82 functions as a sensor controller.

A battery 102 is a lithium-ceramic type battery, and charging and discharging thereof are controlled by a power supply controller 100. The power supply controller 100 supplies the electric power from the battery 102 to the circuits in the BE device 14 during a battery operation. During non-operation of the battery, the electric power supplied from the FE device or the electric power supplied from the AC adaptor is supplied to the circuits in the BE device 14. Reference numeral 104 shows a power supply line via the AC adaptor.

The BE device 14 controls the FE device, sequentially processes beam data sent from the FE device to generate an ultrasound image, and displays the ultrasound image on the touch panel monitor 78. In this process, an operation graphic image is also displayed with the ultrasound image. In a normal real-time operation, the FE device and the BE device 14 are electrically connected to each other wirelessly or with a wire, and, while the devices are synchronized, the ultrasound diagnosis operation is continuously executed. In the freeze state, in the FE device, operations of the transmission signal generator circuit and the reception signal generator circuit are stopped, and operation of a voltage boosting circuit in the power supply controller 100 is also stopped. In the BE device 14, a static image display is set at the time of freeze, and the content is maintained. Alternatively, the BE device 14 may be configured such that an external display may be connected to the BE device 14.

### (4) Communication Schemes

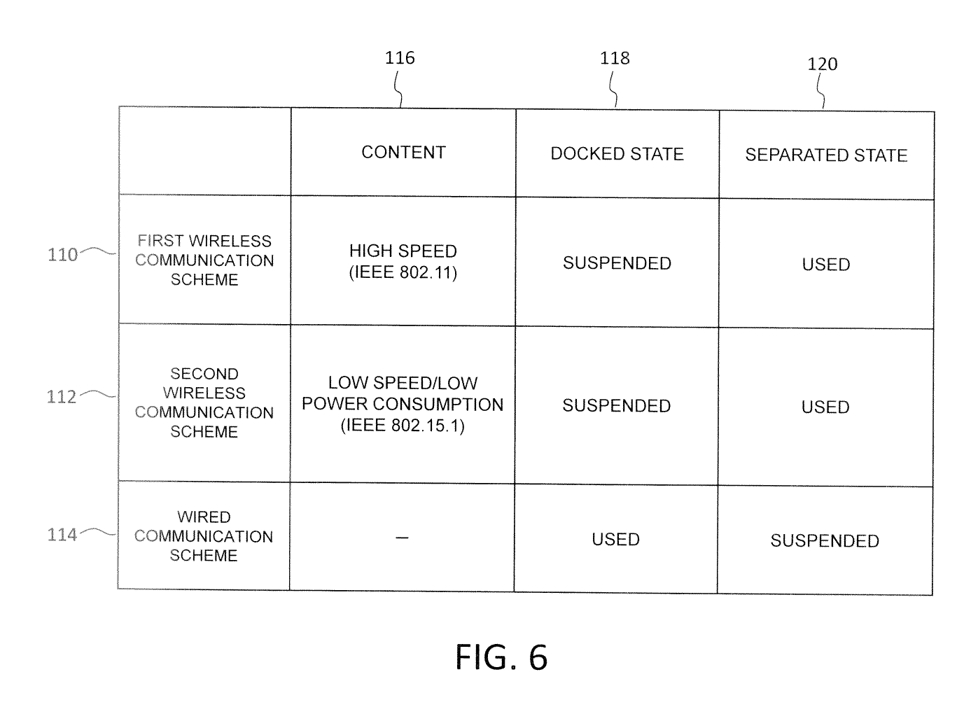

FIG. 6 collectively shows communication schemes used in a docked state 118 and a separated state 120. Reference numeral 110 shows the first wireless communication scheme, and reference numeral 112 shows the second wireless communication scheme. Reference numeral 114 shows the wired communication scheme. Reference numeral 116 shows contents of the wireless communication scheme. In the docked state 118, the wired communication is selected, and the first wireless communicator and the second wireless communicator are set in an operation suspended state in the FE device and the BE device. With such a configuration, electric power can be saved. On the other hand, in the separated state 120, the wireless communication is selected, and the first wireless communicator and the second wireless communicator operate in the FE device and the BE device. In this process, the wireless communication system is set in an operation suspended state. The first wireless communication scheme 110 is faster than the second wireless communication scheme 112. In other words, the second wireless communication scheme 112 is slower than the first wireless communication scheme 110, but is simpler and less expensive, and consumes less power. As the wired communication scheme, a TCP/IP protocol over Ethernet (Registered Trademark) may be exemplified. As the first wireless communication scheme, IEEE 802.11 may be exemplified, and, as the second wireless communication scheme, IEEE 802.15.1 may be exemplified. These are merely exemplary, and other communication schemes may be used. In either scheme, a secure communication scheme is desirably used.

In the present embodiment, the wireless communicator according to the second wireless communication scheme 112 has a function to automatically vary transmission power according to reception intensity (that is, distance). In other words, when the BE device comes near the FE device, control to reduce the transmission power is automatically executed in both devices. Therefore, it is possible to judge that the devices have moved close to each other based on the set transmission power. Alternatively, it is also possible to judge that the two devices are moved close to each other based on the reception intensity, a reception error rate, or the like, in place of the transmission power. As a further alternative, a proximity sensor may be used.

### (5) Setup for Wireless Communication and Establishment of Wireless Communication

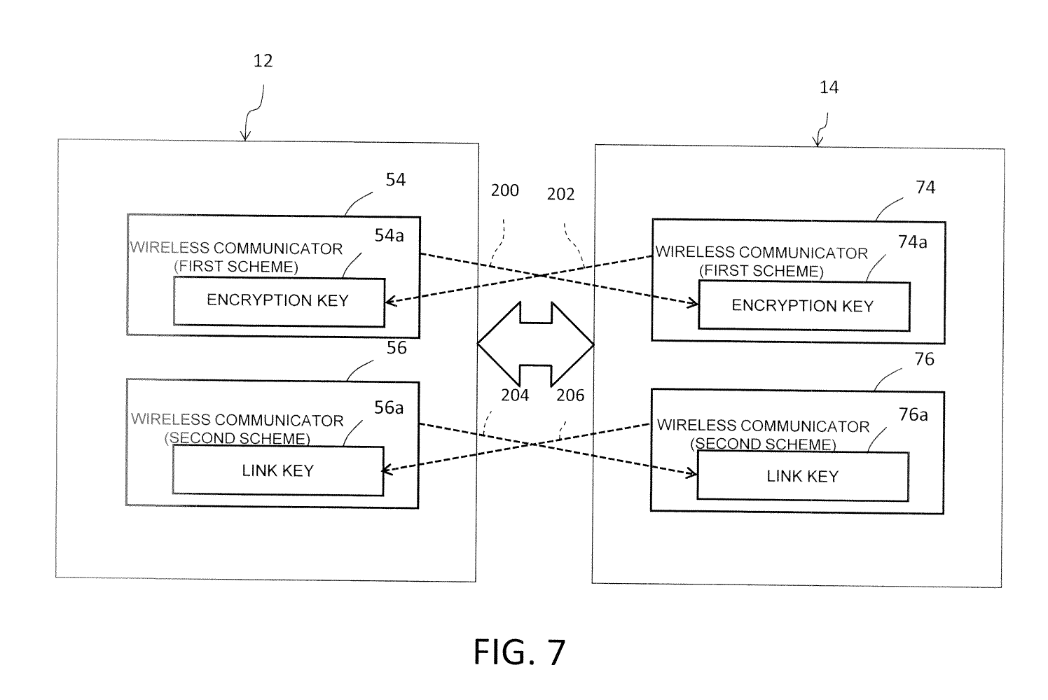

FIG. 7 is a diagram for explaining setup for wireless communication. The setup is performed at the time of shipping from the factory (initial system formation), and also during maintenance, as necessary. In the setup, the plurality of devices of the ultrasound diagnostic system, that is, the FE device 12 and BE device 14 which cooperate in the ultrasound diagnosis, are paired. That is, in order for the reception data transfer and control signal transfer to only be performed between two particular devices 12 and 14, and not with other devices, in other words, in order for the individual devices 12 and 14 to not be wirelessly connected to other unspecified devices, devices for connection are registered in the devices 12 and 14. In actual practice, key information is exchanged between the devices 12 and 14. Because a configuration may be conceived in which a plurality of ultrasound diagnostic systems exist within a wireless communication range, the pairing is also important from the viewpoint of assuring soundness of the system operation.

If the communication is not an internal communication for the ultrasound diagnosis, and is a general communication, the devices 12 and 14 may be wirelessly communicated with other devices. For example, the BE device 14 may be connected to a wireless LAN in the hospital.

In the present embodiment, during the setup for wireless communication, as shown in FIG. 7, the FE device 12 and the BE device 14 are physically connected into a docked state, that is, a wired connection state, and in this state, the key information for exclusive wireless communication is exchanged between the FE device 12 and the BE device 14 (pairing). The key information acquired by the devices 12 and 14 may be the same or different from each other. In the present embodiment, a plurality of key information for a plurality of wireless communication schemes are exchanged. For example, with regard to the first wireless communication scheme, as shown by an arrow 200, an encryption key is transferred through wired communication from the wireless communicator 54 to the wireless communicator 74, and is stored in a memory 74a in the wireless communicator 74. At the same time, as shown by an arrow 202, an encryption key is transferred through the wired communication from the wireless communicator 74 to the wireless communicator 54, and is stored in a memory 54a in the wireless communicator 54. Similarly, with regard to the second wireless communication scheme, as shown by an arrow 204, a link key (for example, PIN code) is transferred through wired communication from the wireless communicator 56 to the wireless communicator 76, and authentication data thereof is stored in a memory 76a in the wireless communicator 76. In addition, as shown by an arrow 206, a link key (for example, PIN code) is transferred through the wired communication from the wireless communicator 76 to the wireless communicator 56, and is stored in a memory 56a in the wireless communicator 56.

The key information is data for establishing wireless communication between two particular devices, and for executing an exclusive wireless communication. The key information may alternatively be understood as authentication data. For the pairing, a device ID, an IP address, a network ID (SSID), and other information may be exchanged. All or a part of these data may be exchanged every time the wireless communication is established. Each of the memories 54a, 56a, 74a, and 76a is formed as a non-volatile memory. Even in a powered-off state, the key information is maintained. Alternatively, individual key information may be stored in a non-volatile memory existing outside the wireless communicator.

At the time of shipping of the ultrasound diagnostic system from the factory, two particular devices 12 and 14 are set in the docked state, and pairing is executed to exchange the key information for the wireless communication utilizing the wired communication. In addition to at the time of shipping from the factory, the pairing may be executed as necessary during maintenance of the ultrasound diagnostic system placed in the hospital or when the ultrasound diagnostic system is re-constructed. At the time of the first wired communication, setup for the wired communication is executed as necessary.

According to the pairing for wireless communication as described above, because the wireless communication can be executed only between two devices that are actually docked, it is possible to prevent a situation where one of the devices is unintentionally connected to other devices. For example, even when a plurality of FE devices and a plurality of BE devices exist in the hospital, only the wireless communication between the paired devices is executed.

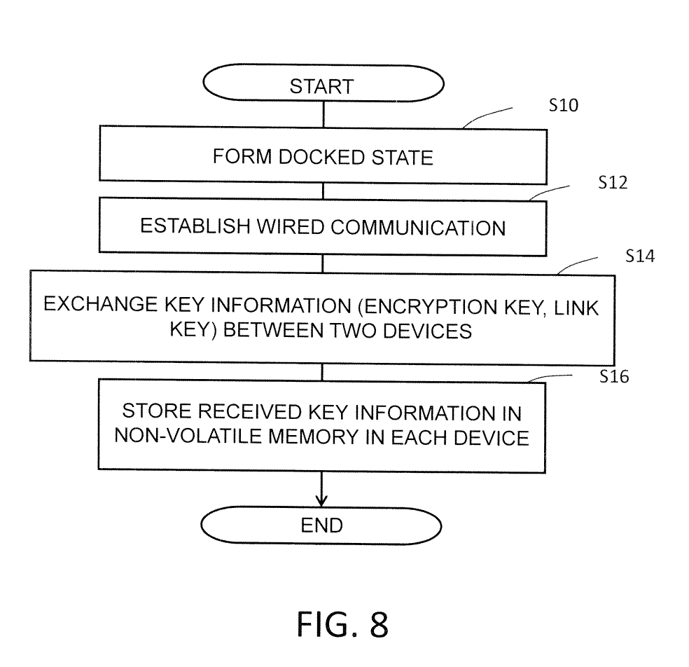

FIG. 8 is a flowchart showing an operation during the setup for wireless communication. In S10, the FE device and the BE device to be paired are set in the docked state. This is done manually. Prior to this process or after this process, a system operation mode is set to a setup (maintenance) mode. In S12, wired communication is established between the two devices. In S14, key information is exchanged between the two devices, and in S16, in each device, the received key information is stored in the non-volatile memory. The exchange of the key information is executed for each wireless communication scheme. In the embodiment, the encryption key and the link key are exchanged.

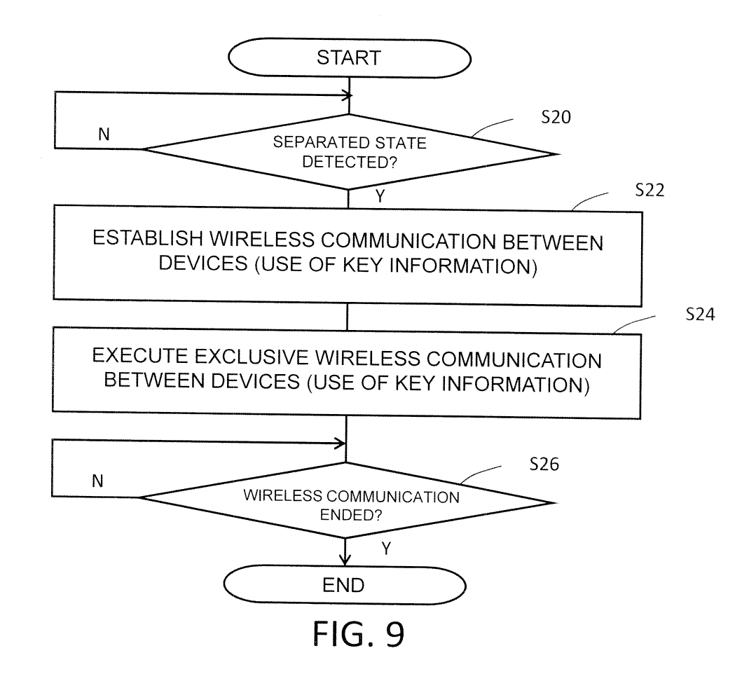

FIG. 9 is a flowchart showing an operation when the wireless communication is established. In S20, it is judged whether or not the separated state is set, that is, whether or not the devices are released from the docked state. If the devices are set in the separated state, S22 is executed. In reality, at the time the devices are set in the separated state, the ultrasound diagnostic system is set in the freeze state, and execution of S22 becomes possible by the user's operation to release freeze. In S22, an authentication process for establishing two systems of wireless communications are executed in each device. In this process, the key information is used in each device. Alternatively, as necessary, authentication data and general data may be exchanged at this stage. In S24, the wireless communications of two systems are actually executed. In this case, other devices cannot be freely connected to the FE device and the BE device, that is, exclusive control is executed for the wireless connection. In S26, it is judged whether or not the wireless communication is to be ended. For example, when the docked state is formed (strictly speaking, when it is judged that a state immediately before the docked state is established), the wireless communication is ended and the devices are set in the freeze state. The operation shown in FIG. 9 is executed every time a new docked state is formed.

According to the above-described embodiment, it is possible to execute the pairing task while actually identifying and checking the pairing target. Therefore, it is possible to reliably prevent pairing in an erroneous relationship. Once the pairing is established, wireless communication with other devices is limited, that is, exclusive wireless communication is assured, and thus, security and stability can be improved.

1. An ultrasound diagnostic system comprising:
a first device and a second device that operate for ultrasound diagnosis, wherein
the first device and the second device selectively take one of a wired connection state which is a docked state and a wireless connection state which is a separated state,
the first device comprises a first wired communicator and a first wireless communicator;
the second device comprises a second wired communicator that executes wired communication with the first wired communicator, and a second wireless communicator that executes in parallel a plurality of wireless communication sessions with the first wireless communicator,
during setup, in the docked state, pairing is executed utilizing the wired communication, in which key information necessary for executing the plurality of exclusive wireless communication sessions between the first device and the second device is exchanged,
the key information includes first key information for authenticating the first device at the second device, which is a plurality of pieces of information sent from the first device to the second device for establishing the plurality of wireless communication sessions, and second key information for authenticating the second device at the first device, which is a plurality of pieces of information sent from the second device to the first device for establishing the plurality of wireless communication sessions, and
the first device and the second device are set in a freeze state when a state change from the docked state to the separated state is detected after the pairing, and then, when an operation to release freeze is executed, the plurality of wireless communication sessions are established between the first wireless communicator and the second wireless communicator by the key information.

2. The ultrasound diagnostic system according to claim 1, wherein
the first key information is stored in a non-volatile storage in the second device, and the second key information is stored in a non-volatile storage in the first device.

3. The ultrasound diagnostic system according to claim 1, wherein
each of the first device and the second device includes a unit that detects the state change.

4. The ultrasound diagnostic system according to claim 1, wherein
the plurality of wireless communication sessions include a wireless communication session for transferring ultrasound reception data from the first device to the second device, and a wireless communication session for transferring control data from the second device to the first device.

5. The ultrasound diagnostic system according to claim 1, wherein
the first device is a front-end device having an ultrasound transmission and reception circuit, and
the second device is a back-end device having an ultrasound image display.

6. The ultrasound diagnostic apparatus according to claim 1, wherein
the first device and the second device are set in a freeze state during a process of changing from the separated state to the docked state, before the docked state is formed.

7. The ultrasound diagnostic apparatus according to claim 1, wherein
the setup is executed at a time of shipping from a factory or during maintenance.

8. A method of operating an ultrasound diagnostic system, comprising:
during setup, setting a first device and a second device for ultrasound diagnosis in a docked state;
during the setup, in the docked state, executing pairing, utilizing wired communication, to exchange key information for executing exclusive wireless communication between the first device and the second device; and
after the pairing, establishing exclusive wireless communication using the key information between the first device and the second device every time the first device and the second device are set in a separated state, wherein
the key information includes first key information for authenticating the first device at the second device, which is information sent from the first device to the second device for establishing the wireless communication, and second key information for authenticating the second device at the first device, which is information sent from the second device to the first device for establishing the wireless communication, and
the first device and the second device are set in a freeze state when a state change from the docked state to the separated state is detected after the pairing, and then, when an operation to release freeze is executed, the wireless communication is established between the first wireless communicator and the second wireless communicator by the key information.
